(19) United States
(12) Patent Application Publication
(10) Pub. No.: US 2024/0409506 A1
(43) Pub. Date: Dec. 12, 2024

(54) Compositions and Methods for Labeling of Thioethers

(71) Applicant: Emory University, Atlanta, GA (US)

(72) Inventors: Monika Raj, Atlanta, GA (US); Benjamin Emenike, Atlanta, GA (US)

(21) Appl. No.: 18/721,136

(22) PCT Filed: Dec. 16, 2022
(86) PCT No.: PCT/US2022/081837

§ 371 Date: Jun. 17, 2024

Related U.S. Application Data
(60) Provisional application No. 63/290,696, filed on Dec. 17, 2021.

(51) Int. Cl. C07C 315/04

(52) U.S. Cl. CPC C07C 315/04

(57) ABSTRACT

This disclosure relates to compositions and methods for labeling of thioethers, e.g., S-methyl thioether such as methionine or peptides containing the same. In certain embodiments, this disclosure relates to methods of forming N-(sulfaneylidene)sulfonamide labeled compounds, comprising contacting a compound containing a thioether with a N-sulfonamide or N-(tetrazol-5-yl)-suilfanimine.

### CROSS-REFERENCE TO RELATED APPLICATIONS

This application claims the benefit of U.S. Provisional Application No. 63/290,696 filed Dec. 17, 2021. The entirety of this application is hereby incorporated by reference for all purposes.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 21197PCT.xml. The XML file is 3 KB, was created on Dec. 15, 2022, and is being submitted electronically via the USPTO patent electronic filing system.

### BACKGROUND

The methyl sulfur containing amino acid, methionine, is oxidized to methionine sulfoxide and reduced back to methionine by biological mechanisms. Similar to phosphorylation states, oxidation states of methionine residues can alter cellular process; however, understanding these implications are limited as methods for detecting methionine and methionine oxide using current techniques, such as mass spectroscopy and antibodies, present limitations. Thus, there is a need to identify improvements.

Moskovitz reports the detection and localization of methionine sulfoxide residues of specific proteins in brain tissue by mass spectroscopy analyses. Protein & Peptide Letters, 2014, 21, 52-55.

Suzuki et al. report methionine sulfoxides in serum proteins as potential clinical biomarkers of oxidative stress. Scientific Reports, 2016, 6, 38299.

Sen et al. report the detection and measurement of methionine oxidation in proteins. Curr Protoc Protein Sci, 2017, 87: 14.16.1-14.16.11.

References cited herein are not an admission of prior art.

### SUMMARY

This disclosure relates to compositions and methods for labeling of thioethers, e.g., S-methyl thioether such as methionine or peptides containing the same. In certain embodiments, this disclosure relates to methods of forming N-(sulfaneylidene)sulfonamide labeled compounds, comprising contacting a compound containing a thioether with a N-sulfonamide or N-(tetrazol-5-yl)-sulfanimine.

In certain embodiments, this disclosure relates to methods of labeling a thioether compound comprising contacting a compound containing a thioether with a metal or metal salt, with a compound comprising a N-sulfonamide providing a N-(sulfaneylidene)sulfonamide labeled compound formed at the thioether group.

In certain embodiments, the thioether is a methyl thioether, methionine or a peptide containing methionine. In certain embodiments, the N-sulfonamide is a N-aryl sulfonamide providing a N-(methylsulfaneylidene)arylsulfonamide labeled compound formed at the thioether group. In certain embodiments, the N-arylsulfonamide is a N-benzenesulfonamide providing a N-(methylsulfaneylidene)benzenesulfonamide labeled compound formed at the thioether group.

In certain embodiments, N-(methylsulfaneylidene)benzenesulfonamide labeled compound comprises a label substituent on the benzene ring or conjugated thereto. In certain embodiments, the label is biotin, an aromatic molecule, a fluorescent dye, a second alkynyl group, a ligand, a receptor, an antibody, or an antigen.

In certain embodiments, this disclosure relates to methods of forming N-(sulfaneylidene)sulfonamide labeled compounds, comprising contacting a compound containing a thioether with an N-(iodaneylidene)sulfonamide compound disclosed herein. In certain embodiments, this disclosure relates to forming N-(sulfaneylidene)cyanamide labeled compounds comprising contacting a compound containing a thioether with a N-(iodaneylidene)cyanamide providing a N-(sulfaneylidene)cyanamide. In certain embodiments, the method further comprises contacting the N-(sulfaneylidene)cyanamide with a reactive labeling agent.

In certain embodiments, this disclosure relates to methods of labeling a thioether compound comprising contacting a compound containing a thioether with a compound comprising a N-(iodaneylidene)sulfonamide providing a N-(sulfaneylidene)sulfonamide labeled compound formed at the thioether group.

In certain embodiments, the thioether is methionine or peptides containing methionine.

In certain embodiments, the N-(iodaneylidene)sulfonamide is N-(phenyliodaneylidene)benzenesulfonamide providing a N-(methylsulfaneylidene)benzene sulfonamide labeled compound formed at the thioether group.

In certain embodiments, the N-(methylsulfaneylidene)benzenesulfonamide labeled compound comprises a label substituent on the benzene ring or conjugated thereto.

In certain embodiments, the N-(methylsulfaneylidene)benzenesulfonamide labeled compound is made by the process of mixing labeled benzenesulfonamide with phenyliodanediyl diacetate.

In certain embodiments, the label is biotin, an aromatic molecule, a fluorescent dye, a second alkynyl group, a ligand, a receptor, an antibody, or an antigen.

In certain embodiments, this disclosure relates to methods of labeling a thioether compound comprising contacting a compound containing a thioether with a compound comprising a N-(iodaneylidene)cyanamide providing N-(sulfaneylidene)cyanamide compound formed at the thioether group.

In certain embodiments, this disclosure relates to methods of labeling a thioether compound comprising contacting a compound containing a thioether with a compound comprising a N-(iodaneylidene)cyanamide providing N-(sulfaneylidene)cyanamide compound formed at the thioether group and contacting the N-(sulfaneylidene)cyanamide compound formed at the thioether group with labeled 2-aminoethane-1-thiol providing a N-(4,5-dihydrothiazol-2-yl)-sulfanimine labeled compound.

In certain embodiments, the thioether is methionine or a peptide containing methionine.

In certain embodiments, the N-(iodaneylidene)cyanamide is N-(phenyliodaneylidene) cyanamide providing a N-(methylsulfaneylidene)cyanamide formed at the thioether group. In certain embodiments, N-(phenyliodaneylidene)cyanamide is made by the process of mixing cyanamide with phenyliodanediyl diacetate.

In certain embodiments, the N-(4,5-dihydrothiazol-2-yl)-sulfanimine labeled compound comprises a label substituent on the dihydrothiazol-2-yl ring or conjugated thereto.

In certain embodiments, the label is biotin, an aromatic molecule, a fluorescent dye, a second alkynyl group, a ligand, a receptor, an antibody, or an antigen.

In certain embodiments, this disclosure relates to methods of labeling a thioether compound comprising contacting a compound containing a thioether with a compound comprising a N-(iodaneylidene)cyanamide providing N-(sulfaneylidene)cyanamide compound formed at the thioether group and contacting the N-(sulfaneylidene)cyanamide compound formed at the thioether group with labeled azido compound providing a N-(tetrazol-5-yl)-sulfanimine labeled compound.

In certain embodiments, the thioether is methionine or a peptide containing methionine.

In certain embodiments, the N-(iodaneylidene)cyanamide is N-(phenyliodaneylidene) cyanamide providing a N-(methylsulfaneylidene)cyanamide formed at the thioether group. In certain embodiments, N-(phenyliodaneylidene)cyanamide is made by the process of mixing cyanamide with phenyliodanediyl diacetate.

In certain embodiments, the N-(tetrazol-5-yl)-sulfanimine labeled compound labeled compound comprises a label substituent on the tetrazol-5-yl ring or conjugated thereto.

In certain embodiments, the label is biotin, an aromatic molecule, a fluorescent dye, a second alkynyl group, a ligand, a receptor, an antibody, or an antigen.

In certain embodiments, the method is selective, i.e., the method does not result in labeling of primary thiol groups.

In certain embodiments, this disclosure relates to compounds, compositions, and kits comprising compounds and reagents used in labeling compounds disclosed herein. In certain embodiments, this disclosure relates to compositions comprising reagents and solid surfaces/supports used for making modifications disclosed herein, including intermediates, and products resulting therefrom.

### BRIEF DESCRIPTION OF THE SEVERAL VIEWS OF THE DRAWINGS

FIG. 1 illustrates the formation of N-(sulfaneylidene)benezensulfonamide labeled compounds in a reaction with a peptide comprising methionine.

FIG. 2A illustrates the formation of N-(sulfaneylidene)cyanamide as an intermediate for further thioether labeling methods of peptides comprising methionine.

FIG. 2B illustrates the formation of N-(4,5-dihydrothiazol-2-yl)-sulfanimine and N-(tetrazol-5-yl)-sulfanimine labeled compounds.

FIG. 3 illustrates the formation of an oxidized derivative of embodiments of this disclosure, N-(methyl(oxo)sulfaneylidene)benzenesulfonamide.

FIG. 4A illustrates the labeling of N-(sulfaneylidene)-4-methylbenezensulfonamide derivatized peptides VKEXK (SEQ ID NO: 2) by reacting 4-methylbenzenesulfonamide, chloramine-T (Ch-T), and a copper salt (CuBr) with a peptide VKEMK (SEQ ID NO: 1) having a methylthiol group (methionine amino acid).

FIG. 4B illustrates a mechanism wherein a metal-nitrene is formed.

FIG. 4C illustrates contemplated starting materials for preparing labeled peptides.

FIG. 5A illustrates N-(sulfaneylidene)benezensulfonamide derivates for use in coupling or attaching labels to solid surfaces.

FIG. 5B illustrates reacting N-(sulfaneylidene)benezensulfonamide derivates with triazine providing substituted solid surfaces or labels.

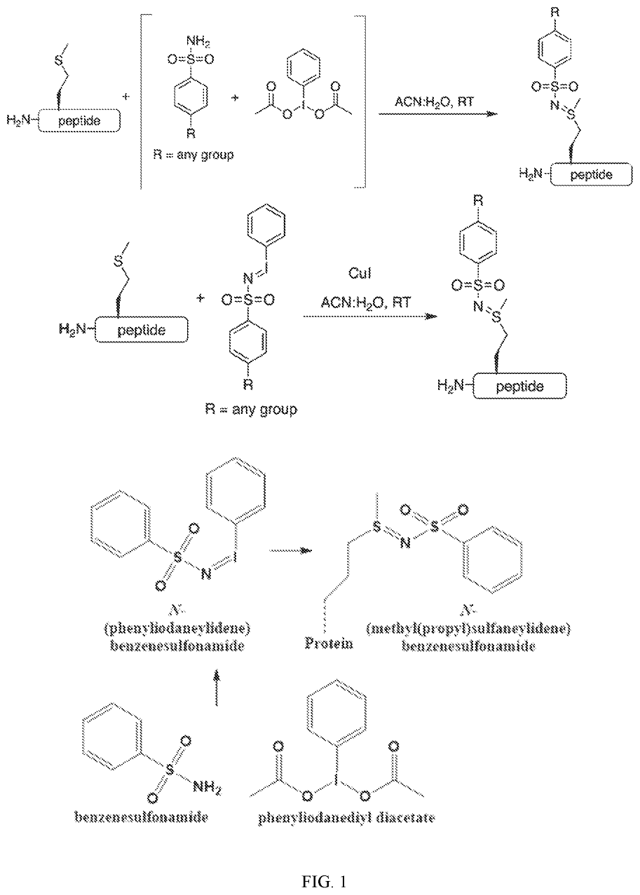

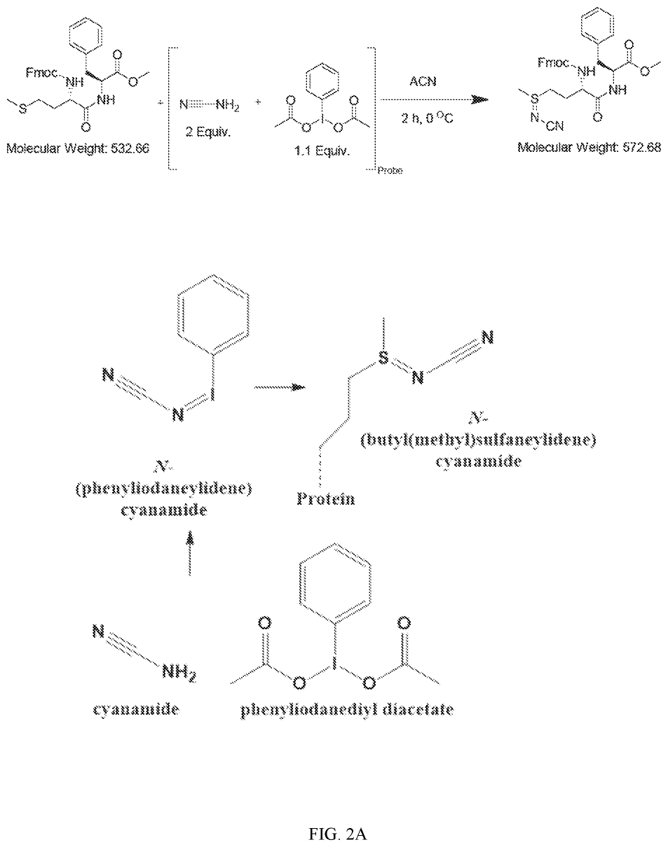

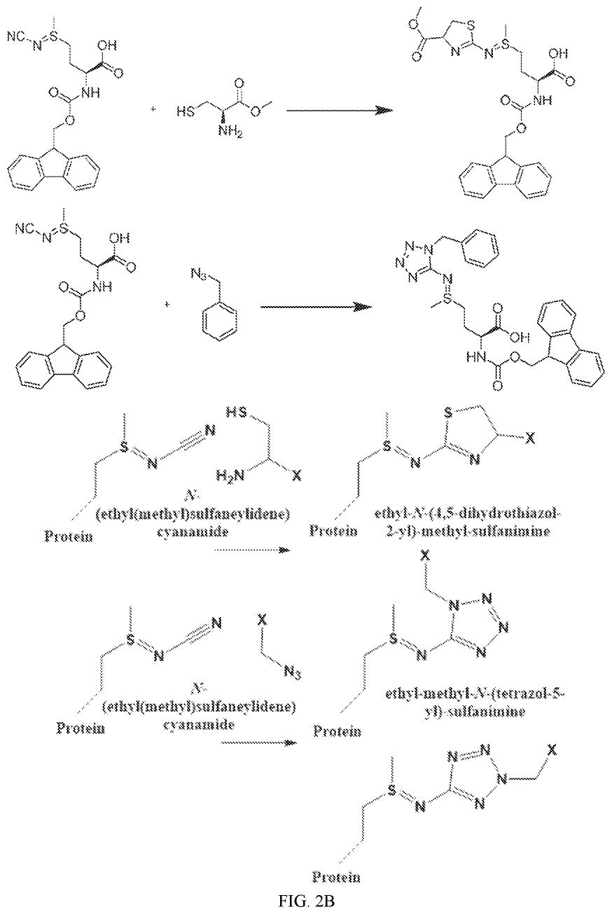

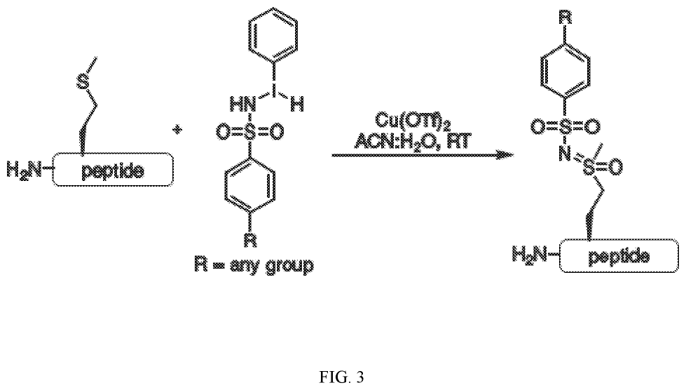

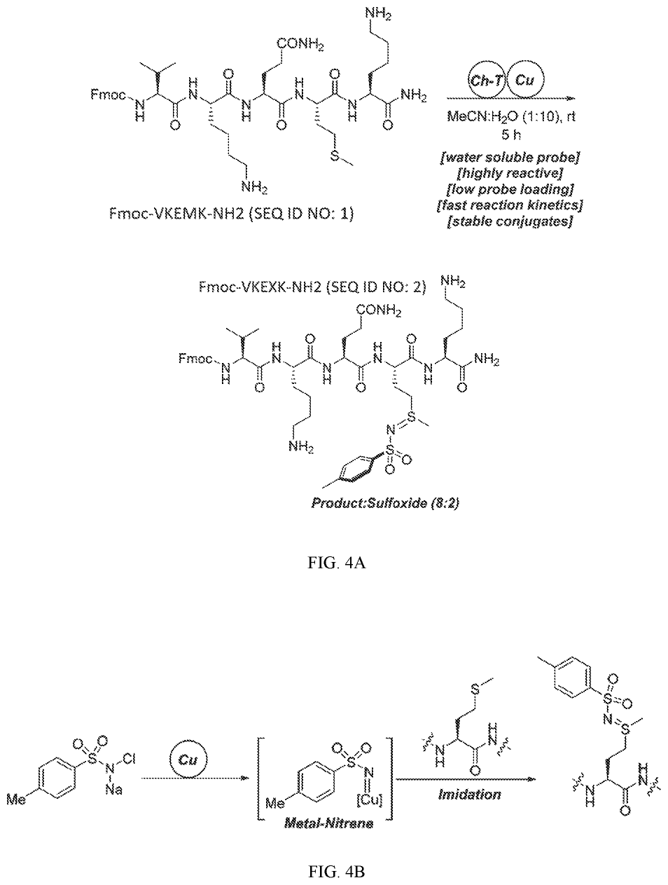

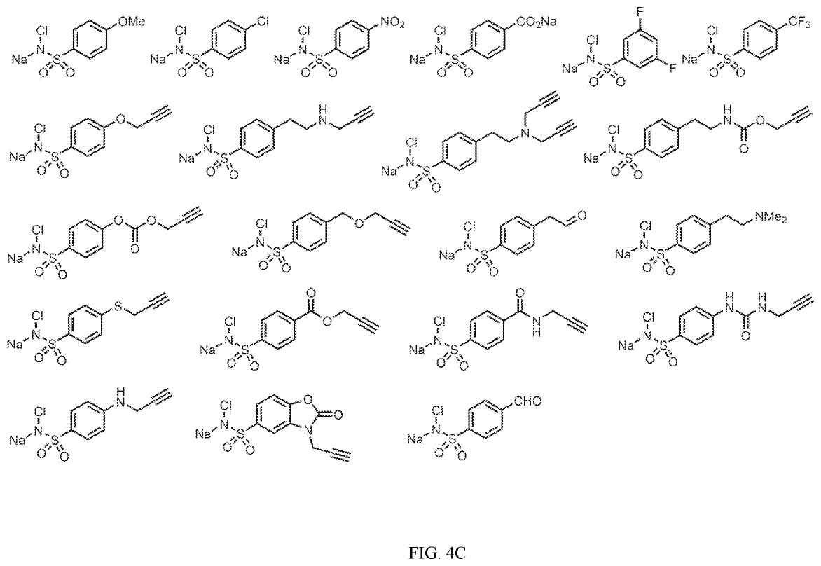

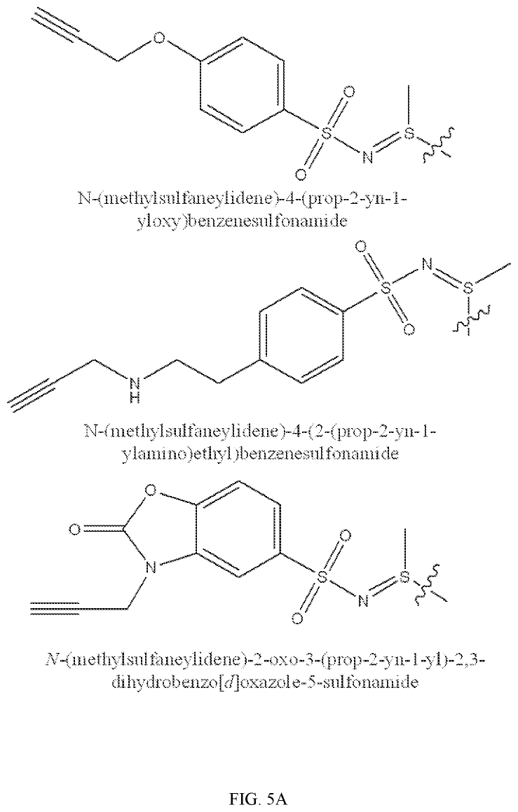

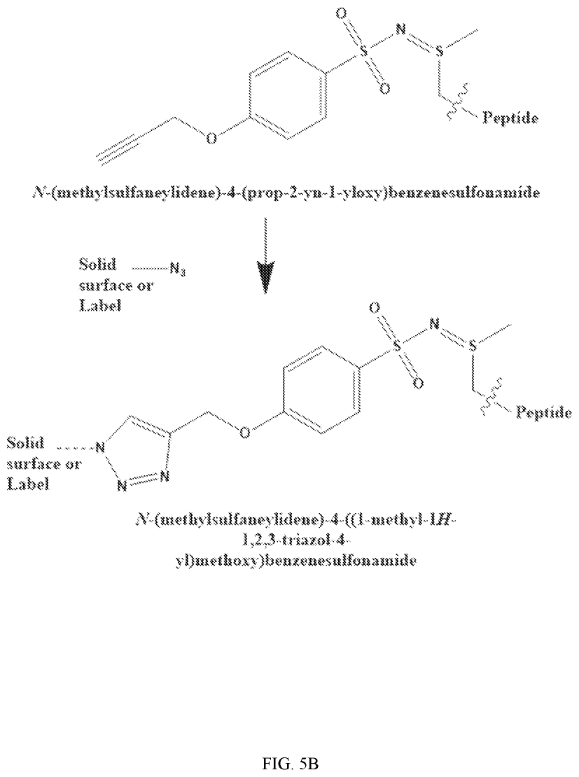

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

An “embodiment” of this disclosure indicates that it is an example and not necessarily limited to such example. Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

The terms “protein,” “peptide,” and “polypeptide” refer to compounds comprising amino acids joined via peptide bonds and are used interchangeably. As used herein, where “amino acid sequence” is recited herein to refer to an amino acid sequence of a protein molecule. An “amino acid sequence” can be deduced from the nucleic acid sequence encoding the protein. However, terms such as “peptide” or “protein” are not meant to limit be limited to natural amino acids. The term includes naturally and non-naturally derived material optionally having naturally or non-naturally occurring amino acids and modifications such as, substitutions, glycosylations, and addition of hydrophilic or lipophilic moieties. In certain embodiments, the protein/peptide/polypeptide comprises more than three, four, five, six, seven, eight, nine, or ten amino acids.

The term “nucleobase polymer” refers to a polymer comprising nitrogen containing aromatic or heterocyclic bases that bind to naturally occurring nucleic acids through hydrogen bonding otherwise known as base pairing. A typical nucleobase polymer is a nucleic acid, RNA, DNA, or chemically modified form thereof. A nucleobase polymer may contain DNA or RNA or a combination of DNA or RNA nucleotides or may be single or double stranded or both, e.g., they may contain overhangs, hairpins, bends, etc. Nucleobase polymers may contain naturally occurring or synthetically modified bases and backbones.

As used herein, the term “conjugated” refers to linking molecular entities through covalent bonds, or by other specific binding interactions, such as due to hydrogen bonding and other van der Walls forces. The force to break a covalent bond is high, e.g., about 1500 pN for a carbon-to-carbon bond. The force to break a combination of strong protein interactions is typically a magnitude less, e.g., biotin to streptavidin is about 150 pN. Thus, a skilled artisan would understand that conjugation must be strong enough to bind molecular entities in order to implement the intended results.

A “linking group” refers to any variety of molecular arrangements that can be used to bridge or conjugate molecular moieties together. An example formula may be —Rₙ— wherein R is selected individually and independently at each occurrence as: —CRₙRₙ—, —CHRₙ—, —CH—, —C—, —CH₂—, —C(OH)Rₙ, —C(OH)(OH)—, —C(OH)H, —C(Hal)Rₙ—, —C(Hal)(Hal)-, —C(Hal)H—, —C(N₃)Rₙ—, —C(CN)Rₙ—, —C(CN)(CN)—, —C(CN)H—, —C(N₃)(N₃)—, —C(N₃)H—, —O—, —S—, —N—, —NH—, —NRₙ—, —(C═O)—, —(C═NH)—, —(C═S)—, —(C═CH₂)—, which may contain single, double, or triple bonds individually and independently between the R groups. If an R is branched with an Rₙ it may be terminated with a group such as —CH₃, —H, —CH═CH₂, —CCH, —OH, —SH, —NH₂, —N₃, —CN, or -Hal, or two branched Rs may form an aromatic or non-aromatic cyclic structure. It is contemplated that in certain instances, the total Rs or “n” may be less than 100 or 50 or 25 or 10. Examples of linking groups include bridging alkyl groups, alkoxyalkyl, and aromatic groups.

The term “specific binding agent” refers to a molecule, such as a proteinaceous molecule, that binds a target molecule with a greater affinity than other random molecules or proteins. Examples of specific binding agents include antibodies that bind an epitope of an antigen or a receptor which binds a ligand. “Specifically binds” refers to the ability of a specific binding agent (such as an ligand, receptor, enzyme, antibody or binding region/fragment thereof) to recognize and bind a target molecule or polypeptide, such that its affinity (as determined by, e.g., affinity ELISA or other assays) is at least 10 times as great, but optionally 50 times as great, 100, 250 or 500 times as great, or even at least 1000 times as great as the affinity of the same for any other or other random molecule or polypeptide.

As used herein, the term “ligand” refers to any organic molecule, i.e., substantially comprised of carbon, hydrogen, and oxygen, that specifically binds to a “receptor.” Receptors are organic molecules typically found on the surface of a cell. Through binding a ligand to a receptor, the cell has a signal of the extra cellular environment which may cause changes inside the cell. As a convention, a ligand is usually used to refer to the smaller of the binding partners from a size standpoint, and a receptor is usually used to refer to a molecule that spatially surrounds the ligand or portion thereof. However as used herein, the terms can be used interchangeably as they generally refer to molecules that are specific binding partners. For example, a glycan may be expressed on a cell surface glycoprotein and a lectin protein may bind the glycan. As the glycan is typically smaller and surrounded by the lectin protein during binding, it may be considered a ligand even though it is a receptor of the lectin binding signal on the cell surface. An antibody may be a receptor, and the epitope may be considered the ligand. In certain embodiments, a ligand is contemplated to be a compound that has a molecular weight of less than 500 or 1,000. In certain embodiments, a receptor is contemplated to be a protein-based compound that has a molecular weight of greater than 1,000, 2,000 or 5,000. In any of the embodiments disclosed herein the position of a ligand and a receptor may be switched.

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a peptide “label” refers to incorporation of a heterologous polypeptide in the peptide, wherein the heterologous sequence can be identified by a specific binding agent, antibody, or bind to a metal such as nickel/nitrilotriacetic acid, e.g., a poly-histidine sequence. Specific binding agents and metals can be conjugated to solid surfaces to facilitate purification methods. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S or ¹³¹I) fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels may be attached by spacer arms of various lengths to reduce potential steric hindrance.

In certain contexts, an “antibody” refers to a protein-based molecule that is naturally produced by animals in response to the presence of a protein or other molecule or that is not recognized by the animal's immune system to be a “self” molecule, i.e., recognized by the animal to be a foreign molecule and an antigen to the antibody. The immune system of the animal will create an antibody to specifically bind the antigen, and thereby targeting the antigen for elimination or degradation. It is well recognized by skilled artisans that the molecular structure of a natural antibody can be synthesized and altered by laboratory techniques. Recombinant engineering can be used to generate fully synthetic antibodies or fragments thereof providing control over variations of the amino acid sequences of the antibody. Thus, as used herein the term “antibody” is intended to include natural antibodies, monoclonal antibody, or non-naturally produced synthetic antibodies, and binding fragments, such as single chain binding fragments. These antibodies may have chemical modifications. The term “monoclonal antibodies” refers to a collection of antibodies encoded by the same nucleic acid molecule that are optionally produced by a single hybridoma (or clone thereof) or other cell line, or by a transgenic mammal such that each monoclonal antibody will typically recognize the same antigen. The term “monoclonal” is not limited to any particular method for making the antibody, nor is the term limited to antibodies produced in a particular species, e.g., mouse, rat, etc.

Hydrophilic polymers contain polar or charged functional groups, rendering them soluble in water. Examples include polyethylene glycol, polylactides, polyglycolide, poly(C-caprolactone), poly(2-methoxyethyl acrylate), poly(tetrahydrofurfuryl acrylate), poly(2-methacryloyloxyethyl phosphorylcholine), poly(p-dioxanone), poly(serine methacrylate), poly[oligo(ethylene glycol) vinyl ether], poly{[2-(methacryloyloxy)ethyl], copolymers of ethylene glycol and propylene glycol, poly(oxyethylated polyol), poly(olefmic alcohol), poly(vinylpyrrolidone), poly(hydroxyalkylmethacrylamide), poly(hydroxyalkylmethacrylate), poly(saccharides), poly(alpha-hydroxy acid), and poly(vinyl alcohol). “PEG,” “polyethylene glycol” and “poly(ethylene glycol)” refers to water-soluble poly(ethylene oxide). Typically, PEGs comprise the following structure “—(OCH₂CH₂)n-” where (n) is 2 to 4000.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, a N-(methyl(oxo)sulfaneylidene)benzenesulfonamide oxidized form, replacing an oxygen atom with a sulfur atom, replacing an amino group with a hydroxyl group, replacing a nitrogen with a protonated carbon (CH) in an aromatic ring, replacing a bridging amino group (—NH—) with an oxy group (—O—), or vice versa. The derivative may be a prodrug. A derivative may be a polypeptide variant. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b}, —NRₐC(═O)NRₐNR_{b}, —NRₐC(═O)OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl.

As used herein, “alkyl” means a noncyclic straight chain or branched, unsaturated or saturated hydrocarbon such as those containing from 1 to 10 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Haloalkyl” refers to an alkyl group wherein one or more or all of the hydrogens are substituted with halogens, e.g., —CH₂CH₂Cl or —CF₃.

“Alkylthio” refers to an alkyl group as defined above with the indicated number of carbon atoms attached through a sulfur bridge. An example of an alkylthio is methylthio, (i.e., —S—CH₃).

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkylamino” refers an alkyl group as defined above with the indicated number of carbon atoms attached through an amino bridge. An example of an alkylamino is methylamino, (i.e., —NH—CH₃).

“Alkanoyl” refers to an alkyl as defined above with the indicated number of carbon atoms attached through a carbonyl bridge (i.e., —(C═O)alkyl).

“Alkylthio” refers to an alkyl group as defined above with the indicated number of carbon atoms attached through a sulfur bridge. An example of an alkylthio is methylthio, (i.e., —S—CH₃).

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkylamino” refers an alkyl group as defined above with the indicated number of carbon atoms attached through an amino bridge. An example of an alkylamino is methylamino, (i.e., —NH—CH₃).

“Aryl” means an aromatic carbocyclic monocyclic or polycyclic ring such as phenyl or naphthyl. Polycyclic ring systems may, but are not required to, contain one or more non-aromatic rings, as long as one of the rings is aromatic.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like

As used herein, “heterocycle” or “heterocyclyl” refers to mono- and polycyclic ring systems having 1 to 4 heteroatoms selected from nitrogen, oxygen, and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

As used herein, “heteroaryl” or “heteroaromatic” refers an aromatic heterocarbocycle having 1 to 4 heteroatoms selected from nitrogen, oxygen, and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isoxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

### Methods and Compositions for Detecting Methionine and Proteins Comprising the Same

This disclosure relates to compositions and methods for labeling of thioethers, e.g., S-methyl thioether such as methionine or peptides or proteins containing the same. In certain embodiments, this disclosure relates to methods of forming N-(sulfaneylidene)sulfonamide labeled compounds, comprising contacting a compound containing a thioether with a N-sulfonamide or N-(tetrazol-5-yl)-sulfanimine.

In certain embodiments, this disclosure relates to methods of labeling a thioether compound comprising contacting a compound containing a thioether, a metal salt, with a compound comprising a N-sulfonamide providing a N-(sulfaneylidene)sulfonamide labeled compound formed at the thioether group.

In certain embodiments, the thioether is a methyl thioether, methionine or a peptide containing methionine. In certain embodiments, the N-sulfonamide is a N-aryl sulfonamide providing a N-(methylsulfaneylidene)arylsulfonamide labeled compound formed at the thioether group. In certain embodiments, the N-arylsulfonamide is a N-benzenesulfonamide providing a N-(methylsulfaneylidene)benzenesulfonamide labeled compound formed at the thioether group.

In certain embodiments, N-(methylsulfaneylidene)benzenesulfonamide labeled compound comprises a label substituent on the benzene ring or conjugated thereto.

In certain embodiments, N-(methylsulfaneylidene)benzenesulfonamide labeled compound is selected from a:
N-(methylsulfaneylidene)-4-methylbenzenesulfonamide;
N-(methylsulfaneylidene)-4-methoxybenzenesulfonamide;
N-(methylsulfaneylidene)-4-chlorobenzenesulfonamide;
N-(methylsulfaneylidene)-4-nitrobenzenesulfonamide;
N-(methylsulfaneylidene)-4-carboxybenzenesulfonamide;
N-(methylsulfaneylidene)-3,5-diflorobenzenesulfonamide;
N-(methylsulfaneylidene)-4-trifluoromethylbenzenesulfonamide;
N-(methylsulfaneylidene)-4-formylbenzenesulfonamide;
N-(methylsulfaneylidene)-4-(prop-2-yn-1-ylamino)benzenesulfonamide
N-(methylsulfaneylidene)-4-(prop-2-yn-1-yloxy)benzenesulfonamide;
N-(methylsulfaneylidene)-4-(prop-2-yn-1-ylthio)benzenesulfonamide;
N-(methylsulfaneylidene)-4-(2-(prop-2-yn-1-ylamino)ethyl)benzenesulfonamide;
N-(methylsulfaneylidene)-4-(2-(di(prop-2-yn-1-yl)amino)ethyl)benzenesulfonamide;
4-(N-(methylsulfaneylidene)sulfamoyl)phenethyl prop-2-yn-1-yl carbamate;
4-(N-(methylsulfaneylidene)sulfamoyl)phenyl prop-2-yn-1-yl carbonate;
N-(methylsulfaneylidene)-4-(2-aminoethyl)-benzenesulfonamide;
N-(methylsulfaneylidene)-4-(2-oxoethyl)benzenesulfonamide;
N-(methylsulfaneylidene)-4-((prop-2-yn-1-yloxy)methyl)benzenesulfonamide;
4-(N-(methylsulfaneylidene)sulfamoyl) prop-2-yn-1-yl benzoate;
4-(N-(methylsulfaneylidene)sulfamoyl)N-(prop-2-yn-1-yl) benzamide; and
N-(methylsulfaneylidene)-2-oxo-3-(prop-2-yn-1-yl)-2,3-dihydrobenzo[d]oxazole-5-sulfonamide, or salt thereof.

In certain embodiments, the label is biotin, an aromatic molecule, a fluorescent dye, a second alkynyl group, a ligand, a receptor, an antibody, or an antigen.

In certain embodiments, this disclosure relates to methods of forming N-(sulfaneylidene)sulfonamide labeled compounds, comprising contacting a compound containing a thioether with N-(iodaneylidene)sulfonamide compound disclosed herein.

In certain embodiments, this disclosure relates to methods of forming N-(sulfaneylidene) sulfonamide labeled compounds comprising contacting a compound containing a thioether, e.g., methionine or peptides or proteins containing the same, with a compound comprising a N-(iodaneylidene)sulfonamide providing a N-(sulfaneylidene)sulfonamide compound formed at the thioether group.

In certain embodiments, the N-(iodaneylidene)sulfonamide is N-(phenyliodaneylidene)benzenesulfonamide compound providing a N-(methylsulfaneylidene)benzenesulfonamide formed at the thioether group.

In certain embodiments, the N-(methylsulfaneylidene)benzenesulfonamide labeled compound comprises a label substituent on the benzene ring or conjugated thereto.

In certain embodiments, the label is biotin, an aromatic molecule, a fluorescent dye, an alkynyl group, a ligand, a receptor, an antibody, or an antigen.

In certain embodiments, the method further comprises contacting the alkynyl labeled compound with a solid surface conjugated to a triazene under conditions such that a triazole compound is conjugated to the solid surface.

In certain embodiments, the method further comprises contacting the triazole compound conjugated to the solid surface with an acid solution such that the purified compound is cleaved from the solid surface providing purified compound.

In certain embodiments, the label is a ligand providing ligand labeled compound. In certain embodiments, the method further comprises contacting the ligand labeled compound with a solid surface conjugated to receptor under conditions such that the ligand labeled compound is conjugated to the solid surface.

In certain embodiments, the label is biotin providing biotin labeled compound. In certain embodiments, the method further comprises contacting the biotin labeled compound with a solid surface conjugated to avidin or streptavidin under conditions such that the biotin labeled compound is conjugated to the solid surface.

In certain embodiments, the N-(phenyliodaneylidene)benzenesulfonamide labeled compound has the following formula,

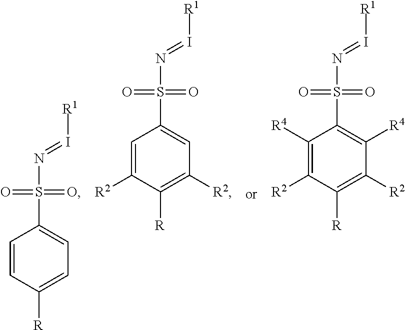

or derivatives thereof, wherein R is optionally substituted to a label or conjugated to a solid support through a linking group. In certain embodiments, R is a hydrogen, alkyl, halogen, haloalkyl, alkoxy, alkylthio, dialkylamino, acetamido, nitrile, nitro, formyl, carboxyl, carbamoyl, or N-substituted carbamoyl group, wherein R is optionally substituted with one or more substituents, or conjugated to a label or a solid support through a linking group. In certain embodiments, R¹ is phenyl, aryl, or heteroaryl, wherein R¹ is optionally substituted. In certain embodiments, R, R² or R⁴ are individually and independently at each occurrence hydrogen, alkyl, halogen, haloalkyl, alkoxy, alkylthio, dialkylamino, acetamido, nitrile, nitro, formyl, carboxyl, carbamoyl, or N-substituted carbamoyl group, wherein R, R² or R⁴ are optionally substituted e.g., conjugated to a label or a solid support through a linking group, or R² and R, or R² and R⁴, and the attached atoms together form an aromatic or non-aromatic ring optionally substituted e.g., conjugated to a label or a solid support through a linking group. In certain embodiments, R is a linking group comprising an alkynyl group or R is —(C═O)NHR⁵—(C═O)OR⁵, —(C═O)SR⁵, or —(C═O)R⁵ wherein R⁵ is an alkynyl group. In certain embodiments, R, R² or R⁴ is a linking group comprising an alkynyl group or R, R² or R⁴ is —(C═O)NHR⁵—(C═O)OR⁵, —(C═O)SR⁵, or —(C═O)R⁵, wherein R⁵ is an alkynyl group.

In certain embodiments, the N-(methylsulfaneylidene)benzenesulfonamide labeled compound has the following formula,

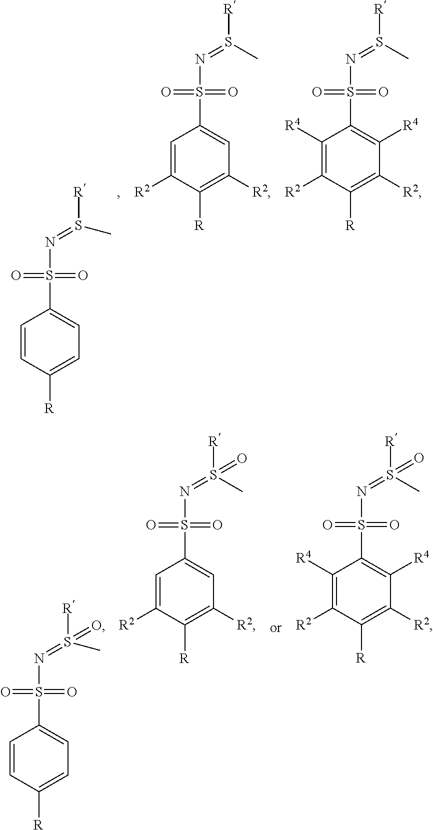

or derivatives thereof, wherein R′ is a modified methionine or peptide or any compound. In certain embodiments, R is optionally substituted to a label or conjugated to a solid support through a linking group. In certain embodiments, R is a hydrogen, alkyl, halogen, haloalkyl, alkoxy, alkylthio, dialkylamino, acetamido, nitrile, nitro, formyl, carboxyl, carbamoyl, or N-substituted carbamoyl group, wherein R is optionally substituted with one or more substituents, or conjugated to a label or a solid support through a linking group. In certain embodiments, R, R² or R⁴ are individually and independently at each occurrence hydrogen, alkyl, halogen, haloalkyl, alkoxy, alkylthio, dialkylamino, acetamido, nitrile, nitro, formyl, carboxyl, carbamoyl, or N-substituted carbamoyl group, wherein R, R² or R⁴ are optionally substituted e.g., conjugated to a label or a solid support through a linking group, or R² and R, or R² and R⁴, and the attached atoms together form an aromatic or non-aromatic ring optionally substituted e.g., conjugated to a label or a solid support through a linking group. In certain embodiments, R is a linking group comprising an alkynyl group or R is —(C═O)NHR⁵—(C═O)OR⁵, —(C═O)SR⁵, or —(C═O)R⁵ wherein R⁵ is an alkynyl group. In certain embodiments, R, R² or R⁴ is a linking group comprising an alkynyl group or R, R² or R⁴ is —(C═O)NHR⁵—(C═O)OR⁵, —(C═O)SR⁵, or —(C═O)R⁵, wherein R⁵ is an alkynyl group.

In certain embodiments, this disclosure relates to forming N-(sulfaneylidene)cyanamide labeled compounds comprising contacting a compound containing a thioether with a N-(iodaneylidene)cyanamide providing a N-(sulfaneylidene)cyanamide. In certain embodiments a N-(iodaneylidene)cyanamide is N-(phenyliodaneylidene)cyanamide. In certain embodiments, this disclosure relates to a thioether or peptide comprising methionine converted to a N-(sulfaneylidene)cyanamide at the thiol group.

In certain embodiments, the method further comprises contacting the N-(sulfaneylidene)cyanamide with a reactive labeling agent.

In certain embodiments, this disclosure relates to forming N-(sulfaneylidene)cyanamide labeled compounds comprising contacting a compound containing a thioether, e.g., methionine or peptides or proteins containing the same, with a compound comprising a N-(iodaneylidene)cyanamide providing N-(sulfaneylidene)cyanamide compound formed at the thioether group.

In certain embodiments the N-(iodaneylidene)cyanamide is N-(phenyliodaneylidene)cyanamide providing a N-(methylsulfaneylidene)cyanamide formed at the thioether group.

In certain embodiments, the method further comprises contacting the N-(sulfaneylidene)cyanamide compound formed at the thioether group with labeled 2-aminoethane-1-thiol providing a N-(4,5-dihydrothiazol-2-yl)-sulfanimine labeled compound.

In certain embodiments, this disclosure relates to compositions, compounds, and peptides or proteins having the following formula:

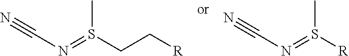

or salts or derivative thereof wherein,

R is a peptide, protein, or compound wherein the peptide, protein, or compound is optionally substituted or conjugated to a label or a solid support through a linking group.

In certain embodiments, the compound is peptide, protein, amino acid, nucleotide, nucleic acid, DNA, RNA, nucleobase polymer, alkyl, aryl, carbocyclyl, heteroaryl optionally substituted with one or more, the same or different substituents such as a halogen, hydroxy, amino, thiol, alkyl, alkoxy, alkylamino, alkylthio, dialkylamino, acetamido, formyl, alkanoyl, carboxyl, carbamoyl, aryl, carbocyclyl or heterocyclyl, or N-substituted carbamoyl group, which is optionally further substituted or conjugated to a label or a solid support through a linking group.

In certain embodiments, the N-(4,5-dihydrothiazol-2-yl)-sulfanimine labeled compound has the following formula

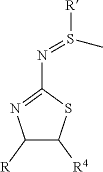

or derivatives thereof, wherein R′ is a modified methionine or peptide or any compound. In certain embodiments, R is optionally substituted to a label or conjugated to a solid support through a linking group. In certain embodiments, R is a hydrogen, alkyl, halogen, haloalkyl, alkoxy, alkylthio, dialkylamino, acetamido, nitrile, nitro, formyl, carboxyl, carbamoyl, or N-substituted carbamoyl group, wherein R is optionally substituted with one or more substituents, or conjugated to a label or a solid support through a linking group. In certain embodiments, R or R⁴ are individually and independently at each occurrence hydrogen, alkyl, halogen, haloalkyl, alkoxy, alkylthio, dialkylamino, acetamido, nitrile, nitro, formyl, carboxyl, carbamoyl, or N-substituted carbamoyl group, wherein R or R⁴ are optionally substituted e.g., conjugated to a label or a solid support through a linking group, or R and R⁴ and the attached atoms together form an aromatic or non-aromatic ring optionally substituted e.g., conjugated to a label or a solid support through a linking group. In certain embodiments, R is a linking group comprising an alkynyl group or R is —(C═O)NHR⁵—(C═O)OR⁵, —(C═O)SR⁵, or —(C═O)R⁵ wherein R⁵ is an alkynyl group. In certain embodiments, R, R² or R⁴ is a linking group comprising an alkynyl group or R, R² or R⁴ is —(C═O)NHR⁵—(C═O)OR⁵, —(C═O)SR⁵, or —(C═O)R⁵, wherein R⁵ is an alkynyl group.

In certain embodiments, the label is biotin, an aromatic molecule, a fluorescent dye, an alkynyl group, a ligand, a receptor, an antibody, or an antigen.

In certain embodiments, the method further comprises contacting the alkynyl labeled compound with a solid surface conjugated to a triazene under conditions such that a triazole compound is conjugated to the solid surface.

In certain embodiments, the method further comprises contacting the triazole compound conjugated to the solid surface with an acid solution such that the purified compound is cleaved from the solid surface providing purified compound.

In certain embodiments, the label is a ligand providing ligand labeled compound. In certain embodiments, the method further comprises contacting the ligand labeled compound with a solid surface conjugated to receptor under conditions such that the ligand labeled compound is conjugated to the solid surface.

In certain embodiments, the label is biotin providing biotin labeled compound. In certain embodiments, the method further comprises contacting the biotin labeled compound with a solid surface conjugated to avidin or streptavidin under conditions such that the biotin labeled compound is conjugated to the solid surface.

In certain embodiments, the method further comprises contacting the N-(sulfaneylidene)cyanamide compound formed at the thioether group with labeled azido compound providing a N-(tetrazol-5-yl)-sulfanimine labeled compound.

In certain embodiments, the N-(tetrazol-5-yl)-sulfanimine labeled compound has the following formula

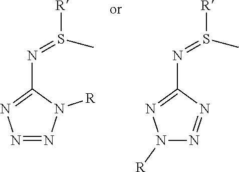

or derivatives thereof, wherein R′ is a modified methionine or peptide or any compound. In certain embodiments, R is optionally substituted to a label or conjugated to a solid support through a linking group. In certain embodiments, R is a hydrogen, alkyl, halogen, haloalkyl, alkoxy, alkylthio, dialkylamino, acetamido, nitrile, nitro, formyl, carboxyl, carbamoyl, or N-substituted carbamoyl group, wherein R is optionally substituted with one or more substituents, or conjugated to a label or a solid support through a linking group. In certain embodiments, R is a linking group comprising an alkynyl group or R is —(C═O)NHR⁵—(C═O)OR⁵, —(C═O)SR⁵, or —(C═O)R⁵ wherein R⁵ is an alkynyl group or a label.

In certain embodiments, the label is biotin, an aromatic molecule, a fluorescent dye, an alkynyl group, a ligand, a receptor, an antibody, or an antigen.

In certain embodiments, the method further comprises contacting the alkynyl labeled compound with a solid surface conjugated to a triazene under conditions such that a triazole compound is conjugated to the solid surface.

In certain embodiments, the method further comprises contacting the triazole compound conjugated to the solid surface with an acid solution such that the purified compound is cleaved from the solid surface providing purified compound.

In certain embodiments, the label is a ligand providing ligand labeled compound. In certain embodiments, the method further comprises contacting the ligand labeled compound with a solid surface conjugated to receptor under conditions such that the ligand labeled compound is conjugated to the solid surface.

In certain embodiments, the label is biotin providing biotin labeled compound. In certain embodiments, the method further comprises contacting the biotin labeled compound with a solid surface conjugated to avidin or streptavidin under conditions such that the biotin labeled compound is conjugated to the solid surface.

In certain embodiments, this disclosure relates to methods and compositions for labeling, isolating, detecting, measuring, and purifying compounds containing thioethers such as methyl thioether, methionine, or protein containing the same. In certain embodiments, this disclosure relates to methods of labeling, isolating, detecting, measuring, and purifying compounds having thioethers such as methyl thioether, methionine, or protein containing the same from a sample optionally utilizing solid supports.

In certain embodiments, the compound for labeling having a thioether is peptide, protein, amino acid, nucleotide, nucleic acid, DNA, RNA, nucleobase polymer, alkyl, aryl, carbocyclyl, heteroaryl optionally substituted with one or more, the same or different substituents such as a halogen, hydroxy, amino, thiol, alkyl, alkoxy, alkylamino, alkylthio, dialkylamino, acetamido, formyl, alkanoyl, carboxyl, carbamoyl, aryl, carbocyclyl or heterocyclyl, or N-substituted carbamoyl group, which is optionally further substituted or conjugated to a label or a solid support through a linking group.

In certain embodiments, methods disclosed herein further comprises contacting the labeled compound or compound conjugated to the solid surface with an acid solution such that the compound is cleaved from the solid surface.

In certain embodiments, for any of the methods disclosed herein the methods further comprise determining the molecular weight or exact mass of the peptide or compound.

In certain embodiments, for any of the methods disclosed herein the methods further comprise separating a purified, cleaved, or isolated peptide composition or compound composition into two or more peptides or proteins or compounds. In certain embodiments, separating is by chromatography. In certain embodiments, washing a solid support containing a labeled peptide provides a purified peptide. In certain embodiments, for any of the methods disclosed herein the washing step is optionally omitted.

In certain embodiments, for any of the methods disclosed herein reactions are in an aqueous solution with a pH of between 6.5 to 8.5. In certain embodiments, for any of the methods disclosed herein reactions are in an aqueous solution with a pH of between 7.0 to 8.5. In certain embodiments, for any of the methods disclosed herein reactions are in an aqueous solution with a pH of between 7.0 to 8.0.

In certain embodiments, for any of the methods disclosed herein reactions are at about room temperature, e.g., between 10 and 30 degrees Celsius. In certain embodiments, for any of the methods disclosed herein reactions are at between 5 and 40 degrees Celsius.

In certain embodiments, this disclosure relates to methods of contacting a thioether compound or protein containing methionine wherein the thioether compound or protein is in a sample such as a biological sample, (e.g., cell, tissue, etc.) or environmental sample. Biological samples may be obtained from animals (including humans) and encompass fluids, blood, solids, tissues, and gases. Environmental samples include environmental material such as surface matter, soil, water, and industrial samples. In certain embodiments, the method does not result in the labeling of thiol, e.g., cysteine in the sample.

In certain embodiments, the compound is peptide, amino acid, nucleotide, nucleic acid, DNA, RNA, nucleobase polymer, alkyl, aryl, carbocyclyl, heteroaryl optionally substituted with one or more, the same or different substituents such as a halogen, hydroxy, amino, thiol, alkyl, alkoxy, alkylamino, alkylthio, dialkylamino, acetamido, formyl, alkanoyl, carboxyl, carbamoyl, aryl, carbocyclyl or heterocyclyl, or N-substituted carbamoyl group, which is optionally further substituted or conjugated to a label or a solid support through a linking group.

In certain embodiments, this disclosure relates to a compound or material comprising or coated with or conjugate to chemical arrangements disclosed herein.

With regard to any of the embodiments disclosed herein, a solid support is a particle, magnetic particle, resin, glass, plastic, silicon, wafer, or multi-well plate (polystyrene microtiter plate).

In certain embodiments, a material, solid support, or particle further comprises or is coated with or conjugated to a hydrophilic polymer.

### Sample Procedure for Protein Labelling:

MeCN and Milli-Q water were degassed through Freeze-Pump-Thaw method for 5 times and used successively. All stock solutions in the method were made with degassed solvents and used immediately. To a 4 mL drum vial was added myoglobin (2 mg, 0.12 μmol) at room temperature. Then the flask was flushed through N₂ and degassed water (700 μL) and MeCN (100 L) was added successively and stirred for 5 min. Then to it, CuBr (10 equiv, 100 μL from the stock solution in MeCN) and probe (10 equiv, 100 μL from the stock solution in H₂O) was added under N₂ successively. The reaction mixture was then stirred for 1 hour and quenched with 50 μL 0.5 N HCl. About 1 mg of protein (500 μL) of the reaction aliquot was taken and it was filtered through Amicon Ultram 0.5 mL 3K MWCO/Thermo-scientific Pierce concentrator PES 3K MWCO 0.5 mL and washed with 8×500 μL of H₂O to remove the small molecules. Finally, the total volume of the solution was adjusted to 500 μL.

For further purification, HPLC analyses were performed on an Agilent 1260 Infinity LCm system using a reverse-phase C18 column (Eclipse Plus™ C18 column, 4.6×100 mm, 5 μm). HPLC method: 0-70% gradient of Buffer B (MeCN+0.1% TFA) over 20 min, followed by 2 min flush at 90% Buffer B, followed by 2 min re-equilibration to 100% buffer A (water+0.1% TFA) (Flow rate: 0.4 mL/min). The collected HPLC fractions was then lyophilized and finally dissolved in 500 μL Buffer B (water+0.1% TFA) and analyzed on Agilent 6545X™ LC/Q-TOF for intact protein analysis.

What is claimed is:

1. A method of labeling a thioether compound comprising contacting a compound containing a thioether, with a metal or salt thereof and compound comprising a N-sulfonamide providing a N-(sulfaneylidene)sulfonamide labeled compound formed at the thioether group.

2. The method of claim 1 wherein the thioether is a methyl thioether, methionine or a peptide containing methionine.

3. The method of claim 1 wherein the N-sulfonamide is a N-aryl sulfonamide providing a N-(methylsulfaneylidene)arylsulfonamide labeled compound formed at the thioether group

4. The method of claim 3 wherein the N-arylsulfonamide is a N-benzenesulfonamide providing a N-(methylsulfaneylidene)benzenesulfonamide labeled compound formed at the thioether group.

5. The method of claim 4, wherein N-(methylsulfaneylidene)benzenesulfonamide labeled compound comprises a label substituent on the benzene ring or conjugated thereto.

6. The method of claim 5, wherein N-(methylsulfaneylidene)benzenesulfonamide labeled compound is selected from a:
N-(methylsulfaneylidene)-4-methylbenzenesulfonamide;
N-(methylsulfaneylidene)-4-methoxybenzenesulfonamide;
N-(methylsulfaneylidene)-4-chlorobenzenesulfonamide;
N-(methylsulfaneylidene)-4-nitrobenzenesulfonamide;
N-(methylsulfaneylidene)-4-carboxybenzenesulfonamide;
N-(methylsulfaneylidene)-3,5-diflorobenzenesulfonamide;
N-(methylsulfaneylidene)-4-trifluoromethylbenzenesulfonamide;
N-(methylsulfaneylidene)-4-formylbenzenesulfonamide;
N-(methylsulfaneylidene)-4-(prop-2-yn-1-ylamino)benzenesulfonamide
N-(methylsulfaneylidene)-4-(prop-2-yn-1-yloxy)benzenesulfonamide;
N-(methylsulfaneylidene)-4-(prop-2-yn-1-ylthio)benzenesulfonamide;
N-(methylsulfaneylidene)-4-(2-(prop-2-yn-1-ylamino)ethyl)benzenesulfonamide;
N-(methylsulfaneylidene)-4-(2-(di(prop-2-yn-1-yl)amino)ethyl)benzenesulfonamide;
4-(N-(methylsulfaneylidene)sulfamoyl)phenethyl prop-2-yn-1-yl carbamate;
4-(N-(methylsulfaneylidene)sulfamoyl)phenyl prop-2-yn-1-yl carbonate;
N-(methylsulfaneylidene)-4-(2-aminoethyl)-benzenesulfonamide;
N-(methylsulfaneylidene)-4-(2-oxoethyl)benzenesulfonamide;
N-(methylsulfaneylidene)-4-((prop-2-yn-1-yloxy)methyl)benzenesulfonamide;
4-(N-(methylsulfaneylidene)sulfamoyl) prop-2-yn-1-yl benzoate;
4-(N-(methylsulfaneylidene)sulfamoyl)N-(prop-2-yn-1-yl) benzamide; and
N-(methylsulfaneylidene)-2-oxo-3-(prop-2-yn-1-yl)-2,3-dihydrobenzo[d]oxazole-5-sulfonamide, or salt thereof.

7. The method of claim 1, wherein the label is biotin, an aromatic molecule, a fluorescent dye, a second alkynyl group, a ligand, a receptor, an antibody, or an antigen.

8. A method of labeling a thioether compound comprising contacting a compound containing a thioether with a compound comprising a N-(iodaneylidene)sulfonamide providing a N-(sulfaneylidene)sulfonamide labeled compound formed at the thioether group.

9. The method of claim 8 wherein the thioether is a methyl thioether, methionine or a peptide containing methionine.

10. The method of claim 8 wherein the N-(iodaneylidene)sulfonamide is N-(phenyliodaneylidene)benzenesulfonamide providing a N-(methylsulfaneylidene)benzene sulfonamide labeled compound formed at the thioether group.

11. The method of claim 10, wherein N-(methylsulfaneylidene)benzenesulfonamide labeled compound comprises a label substituent on the benzene ring or conjugated thereto.

12. The method of claim 10, wherein N-(methylsulfaneylidene)benzenesulfonamide labeled compound is made by the process of mixing labeled benzenesulfonamide with phenyliodanediyl diacetate.

13. The method of claim 8, wherein the label is biotin, an aromatic molecule, a fluorescent dye, a second alkynyl group, a ligand, a receptor, an antibody, or an antigen.

14. A method of labeling a thioether compound comprising contacting a compound containing a thioether with a compound comprising a N-(iodaneylidene)cyanamide providing N-(sulfaneylidene)cyanamide compound formed at the thioether group and contacting the N-(sulfaneylidene)cyanamide compound formed at the thioether group with labeled 2-aminoethane-1-thiol providing a N-(4,5-dihydrothiazol-2-yl)-sulfanimine labeled compound.

15. The method of claim 14 wherein the thioether is methyl thioether, methionine, or a peptide containing methionine.

16. The method of claim 14 wherein the N-(iodaneylidene)cyanamide is N-(phenyliodaneylidene)cyanamide providing a N-(methylsulfaneylidene)cyanamide formed at the thioether group.

17. The method of claim 16, wherein N-(phenyliodaneylidene)cyanamide is made by the process of mixing cyanamide with phenyliodanediyl diacetate.

18. The method of claim 14 wherein the N-(4,5-dihydrothiazol-2-yl)-sulfanimine labeled compound comprises a label substituent on the dihydrothiazol-2-yl ring or conjugated thereto.

19. The method of claim 14, wherein the label is biotin, an aromatic molecule, a fluorescent dye, a second alkynyl group, a ligand, a receptor, an antibody, or an antigen.

20. (canceled)


SEQUENCE LISTING (2 sequences)

SEQ ID NO: 1 - protein, 5 aa, synthetic construct
VKEMK

SEQ ID NO: 2 - protein, 5 aa, synthetic construct
VKEXK